(19) United States
(12) Patent Application Publication
(10) Pub. No.: US 2024/0180915 A1
(43) Pub. Date: Jun. 6, 2024

(54) OIL-IN-WATER EMULSION OF 6-ETHOXY-7-METHOXY-2-(2-METHYLSULFANYLPHENYL)-3,L-BENZOXAZIN-4-ONE

(71) Applicant: SIXERA PHARMA AB, Stockholm (SE)

(72) Inventors: Anders CARLSSON, Stockholm (SE); Maarten DE CHATEAU, Bromma (SE)

(21) Appl. No.: 18/561,108

(22) PCT Filed: Jun. 1, 2022
(86) PCT No.: PCT/EP2022/064944

§ 371 Date: Nov. 15, 2023

(30) Foreign Application Priority Data
Jun. 1, 2021 (SE) 2130150-2

(51) Int. Cl. A61K 31/536; A61K 9/00; A61K 9/107; A61K 47/10; A61K 47/12; A61K 47/14

(52) U.S. Cl. CPC A61K 31/536; A61K 9/0014; A61K 9/107; A61K 47/10; A61K 47/12; A61K 47/14

(57) ABSTRACT

The present invention provides topical formulations of 6-ethoxy-7-methoxy-2-(2-methylsulfanylphenyl)-3,1-benzoxazin-4-one in the form of oil-in-water emulsions and their use in the in prophylaxis, prevention, and/or treatment of inflammatory skin diseases, especially Netherton syndrome.

### FIELD OF THE INVENTION

The present invention relates to a topical formulation of 6-ethoxy-7-methoxy-2-(2-methylsulfanylphenyl)-3,1-benzoxazin-4-one and its use in the treatment of inflammatory skin diseases, especially Netherton syndrome.

### BACKGROUND OF THE INVENTION

KLK7 (hK7, or stratum corneum chymotryptic enzyme, SCCE) is a serine protease of the kallikrein gene family displaying a chymotrypsin like activity. KLK7 is mainly expressed in the skin and appears to play an important role in skin physiology (Egelrud. 1993. Purification and preliminary characterization of stratum corneum chymotryptic enzyme: a proteinase that may be involved in desquamation. J. Invest. Dermatol. 101, 200-204; Yousef et al. 2000. The KLK7 (PRSS6) gene, encoding for the stratum corneum chymotryptic enzyme is a new member of the human kallikrein gene family—genomic characterization, mapping, tissue expression and hormonal regulation. Gene 254, 119-1281).

KLK7 is involved in the degradation of the intercellular cohesive structure in cornified squamous epithelia in the process of desquamation. The desquamation process is well regulated and delicately balanced with the de novo production of corneocytes to maintain a constant thickness of the stratum corneum. In this regard, KLK7 is reported to be able to cleave the corneodesmosomal proteins corneodesmosin and desmocollin 1 (Simon et al. 2001. Refined characterization of comeodesmosin proteolysis during terminal differentiation of human epidermis and its relationship to desquamation. J. Biol. Chem. 276, 20292-20299; Caubet et al. 2004. Degradation of corneodesmosome proteins by two serine proteases of the kallikrein family, SCTE/KLK5/hK5 and SCCE/KLK7/hK7. J. Invest. Dermatol. 122, 1235-1244; Brattsand et al. 2005. A proteolytic cascade of kallikreins in the stratum corneum. J. Invest. Dermatol. 124, 198-203). In addition, it has been shown that the two lipid processing enzymes β-glucocerebrosidase and acidic sphingomyelinase can be degraded by KLK7 (Hachem et al. 2005. Sustained serine proteases activity by prolonged increase in pH leads to degradation of lipid processing enzymes and profound alterations of barrier function and stratum corneum integrity. J. Invest. Dermatol. 125, 510-520). Both lipid processing enzymes are co-secreted with their substrates glucosylceramides and sphingomyelin and process these polar lipid precursors into their more non-polar products, e.g. ceramides, which are subsequently incorporated into the extracellular lamellar membranes. The lamellar membrane architecture is critical for a functional skin barrier. Finally, KLK7 has been shown to activate the pro-inflammatory cytokine Pro-interleukin-1β (IL-1B) (Nylander-Lundqvist & Egelrud. 1997. Formation of active IL-1β from pro-IL-1β catalyzed by stratum corneum chymotryptic enzyme in vitro. Acta Derm. Venereol. 77, 203-206).

Several studies link an increased activity of KLK7 to inflammatory skin diseases like atopic dermatitis, psoriasis, and Netherton syndrome. An increased KLK7 activity might lead to an uncontrolled degradation of corneodesmosomes resulting in a miss-regulated desquamation, an enhanced degradation of lipid processing enzymes resulting in a disturbed lamellar membrane architecture, or an uncontrolled activation of the pro-inflammatory cytokine IL-1B. It has previously been demonstrated that this could lead to an impaired skin barrier function and inflammation (WO 2004/108139).

The KLK7 activity is controlled on several levels. Various factors might be responsible for an increased KLK7 activity in inflammatory skin diseases. Firstly, the amount of protease being expressed might be influenced by genetic factors. Such a genetic link, a polymorphism in the 3′-UTR in the KLK7 gene, has been described (Vasilopoulos et al. 2004. Genetic association between an AACC insertion in the 3′UTR of the stratum corneum chymotryptic enzyme gene and atopic dermatitis. J. Invest. Dermatol. 123, 62-66.). Secondly, since KLK7 is secreted via lamellar bodies to the stratum corneum extracellular space as zymogen and it is not able to autoactivate, it needs to be activated by another protease, e.g. KLK5 (Caubet et al. supra). Uncontrolled activity of such an activating enzyme might result in an over activation of KLK7. Thirdly, activated KLK7 can be inhibited by natural inhibitors like LEKTI, ALP or elafin (Schechter et al. 2005. Inhibition of human kallikreins 5 and 7 by the serine protease inhibitor lympho-epithelial Kazal-type inhibitor (LEKTI). Biol. Chem. 386, 1173-1184; Franzke et al. 1996. Antileukoprotease inhibits stratum corneum chymotryptic enzyme—Evidence for a regulative function in desquamation. J. Biol. Chem. 271, 21886-21890). The decreased expression or the lack of such inhibitors might result in an enhanced activity of KLK7.

It has been found that mutations in the spink gene, coding for LEKTI, are causative for Netherton syndrome (Descargues et al. 2005. Spink5-deficient mice mimic Netherton syndrome through degradation of desmoglein 1 by epidermal protease hyperactivity. Nat. Genet. 37, 56-65) and a single point mutation in the gene is linked to atopic dermatitis (Walley et al. 2001. Gene polymorphism in Netherton and common atopic disease. Nat. Genet. 29, 175-178; Nishio et al. 2003. Association between polymorphisms in the SPINK5 gene and atopic dermatitis in the Japanese. Genes Immun. 4, 515-517).

The hypothesis that an increased activity of KLK7 is linked to inflammatory skin diseases is supported by the following studies: Firstly, Netherton syndrome patients show a phenotype dependent increase in serine protease activity, a decrease in corneodesmosomes, a decrease in the lipid processing enzymes β-glucocerebrosidase and acidic sphingomyelinase, and an impaired barrier function (Descargues et al. 2006. Corneodesmosomal cadherins are preferential targets of stratum corneum trypsin- and chymotrypsin-like hyperactivity in Netherton syndrome. J. Invest. Dermatol. 126, 1622-1632; Hachem et al. 2006. Serine protease activity and residual LEKTI expression determine phenotype in Netherton syndrome. J. Invest. Dermatol. 126, 1609-1621.). Secondly, a transgenic mice overexpressing KLK7 shows a skin phenotype similar to that found in patients with atopic dermatitis (Hansson et al. 2002. Epidermal Overexpression of Stratum Corneum Chymotryptic Enzyme in Mice: A Model for Chronic Itchy Dermatitis. J. Invest. Dermatol. 118, 444-449; Ny & Egelrud. 2003. Transgenic mice over-expressing a serine protease in the skin: evidence of interferon gamma-independent MHC II expression by epidermal keratinocytes. Acta Derm. Venereol. 83, 322-327; Ny & Egelrud. 2004. Epidermal hyperproliferation and decreased skin barrier function in mice overexpressing stratum corneum chymotryptic enzyme. Acta Derm. Venereol. 84, 18-22). Thirdly, in the skin of atopic dermatitis and psoriasis patients elevated levels of KLK7 were described (Ekholm & Egelrud. 1999. Stratum corneum chymotryptic enzyme in psoriasis. Arch. Dermatol. Res. 291, 195-200). Therefore, KLK7 is considered to be a target for the treatment of inflammatory skin diseases like atopic dermatitis, psoriasis or Netherton syndrome and there is a need for specific inhibitors thereof.

WO 2004/108139 describes certain substituted benzoxazinone and thienoxazinone compounds as inhibitors of KLK7.

WO 2015/112081 identifies 6-Ethoxy-7-methoxy-2-(2-methylsulfanylphenyl)-3,1-benzoxazin-4-one as a specific inhibitor of KLK7.

### DESCRIPTION OF THE INVENTION

6-Ethoxy-7-methoxy-2-(2-methylsulfanylphenyl)-3,1-benzoxazin-4-one has low solubility in water but can be dissolved in various organic solvents. However, such organic solvents are unsuitable for use in topical formulations for treatment of skin disease.

Initial attempts to prepare formulations of 6-ethoxy-7-methoxy-2-(2-methylsulfanylphenyl)-3,1-benzoxazin-4-one using common pharmaceutically acceptable solvents and excipients demonstrated a clear tendency for 6-ethoxy-7-methoxy-2-(2-methylsulfanylphenyl)-3,1-benzoxazin-4-one to form needle shaped crystals upon storage of the formulation. Accordingly, further development was needed to arrive at a stable topical formulation where crystal formation of 6-ethoxy-7-methoxy-2-(2-methylsulfanylphenyl)-3,1-benzoxazin-4-one can be avoided.

The present inventors have identified stable topical formulations where crystallization of 6-ethoxy-7-methoxy-2-(2-methylsulfanylphenyl)-3,1-benzoxazin-4-one can be avoided, even after prolonged storage at room temperature.

One aspect of the present invention relates to a topical formulation comprising 6-ethoxy-7-methoxy-2-(2-methylsulfanylphenyl)-3,1-benzoxazin-4-one in an oil-in-water emulsion.

The formulation can comprise from about 0.01% (w/w) to about 0.2% (w/w), such as about 0.025% (w/w) to about 0.1% (w/w) 6-ethoxy-7-methoxy-2-(2-methylsulfanylphenyl)-3,1-benzoxazin-4-one.

Preferably the formulation comprises about 0.075% (w/w) 6-ethoxy-7-methoxy-2-(2-methylsulfanylphenyl)-3,1-benzoxazin-4-one, which is equal to 0.75 mg/g formulation.

The formulation can comprise from about 60% (w/w) to about 75% (w/w) oil phase. Preferably the formulation comprises 70% (w/w) oil phase.

The oil phase can comprise triglyceride oils. The triglyceride oil is preferably a medium chain triglyceride oil (MCT).

Preferably the oil phase comprises more than 50% (w/w) MCT, such as more than 60% (w/w) MCT, or more than 70% (w/w) MCT.

The formulation may further contain other pharmaceutically acceptable excipients such as emollients, emulsion forming agents/emulsifiers, emulsion thickeners and stabilizers, antioxidants, preservatives, and neutralizing/pH modifying agents.

The emollient which may be used in the formulations according to the invention include conventional emollients, such as, for example, isopropyl myristate (IPM), isopropyl palmitate and isopropyl isostearate. Preferably the emollient is isopropyl myristate (IPM).

The emulsion forming agents/emulsifiers which may be used in the formulations according to the invention include conventional emulsion forming agents/emulsifiers, such as, for example, cetomacrogol 1000 (PEG 20 cetostearyl ether), PEG 20 stearyl ether and PEG 2 stearyl ether. Preferably the emulsion forming agent/emulsifier is cetomacrogol 1000 (PEG 20 cetostearyl ether).

The emulsion thickeners and stabilizers which may be used in the formulations according to the invention include conventional emulsion thickeners and stabilizers, such as, for example, cetostearyl alcohol, cetyl alcohol and stearyl alcohol. Preferably the emulsion thickener is cetostearyl alcohol.

The antioxidants which may be used in the formulations according to the invention include conventional antioxidants, such as, for example, a-tocopherol, butylated hydroxy toluene (BHT), butylated hydroxy anisole (BHA), tertiary butyl hydroquinone and propyl gallate. Preferably the antioxidant is butylated hydroxy toluene (BHT).

The preservatives which may be used in the formulations according to the invention include conventional preservatives, such as, for example, sorbic acid, potassium sorbate, benzoic acid, sodium benzoate, benzyl alcohol, phenoxyethanol, benzalkonium chloride, para-hydroxybenzoic acids (parabens), such as methylparaben, ethylparaben, propylparaben, butylparaben, or mixtures thereof such as Phenonip. Preferably the preservative system is a mixture of sorbic acid and potassium sorbate.

The neutralizing/pH modifying agents which may be used in the formulations according to the invention include conventional neutralizing/pH modifying agents, such as, for example buffered aqueous solutions. Preferably the buffered aqueous solution is a citrate buffer, preferably with pH 4.2.

In one embodiment the present invention provides a topical formulation being an oil-in-water emulsion, said formulation comprising,
a) from about 0.01% (w/w) to about 0.2% (w/w), such as about 0.025% (w/w) to about 0.1% (w/w) 6-ethoxy-7-methoxy-2-(2-methylsulfanylphenyl)-3,1-benzoxazin-4-one,
b) from about 60% (w/w) to about 75% (w/w) oil phase,
c) from about 25% (w/w) to about 40% (w/w) aqueous phase, wherein the oil phase comprises more than 50% (w/w) MCT.

In one embodiment the present invention provides a topical formulation being an oil-in-water emulsion, said formulation comprising,
a) about 0.075% (w/w) 6-ethoxy-7-methoxy-2-(2-methylsulfanylphenyl)-3,1-benzoxazin-4-one,
b) about 50% (w/w) MCT,
c) about 10% (w/w) IPM,
d) about 5% (w/w) cetostearyl alcohol,
e) about 5% (w/w) polyethylene glycol hexadecyl ether, and
f) about 30% (w/w) citrate buffer.

In another embodiment the present invention provides a topical formulation being an oil-in-water emulsion, said formulation comprising,
a) about 0.075% (w/w) 6-ethoxy-7-methoxy-2-(2-methylsulfanylphenyl)-3,1-benzoxazin-4-one,
b) about 50% (w/w) MCT,
c) about 5% (w/w) IPM,
d) about 7.5% (w/w) cetostearyl alcohol,
e) about 7.5% (w/w) polyethylene glycol hexadecyl ether, and
f) about 30% (w/w) citrate buffer.

The two formulations described above, being oil-in-water emulsions, comprise to about 70% (w/w) oil phase and about 30% (w/w) aqueous phase. The emulsions as a whole comprise about 50% (w/w) MCT, which corresponds to an oil phase comprising about 71% (w/w) MCT. The amount of emollients, emulsion forming agents/emulsifiers, emulsion thickeners and stabilizers used in the formulation herein may be part of the amount of oil phase.

Another aspect of the present invention provides a topical formulation according to the invention for use in prophylaxis, prevention and/or treatment of a skin disease.

Another aspect of the present invention provides a method for the prophylaxis, prevention and/or treatment of a skin disease which comprises the administration of a topical formulation according to the invention, to a subject in need of such treatment.

The skin disease may be an inflammatory skin disease. The skin inflammatory disease can be selected from Netherton syndrome, atopic dermatitis, contact dermatitis, eczema, psoriasis, acne, epidermal hyperkeratosis, acanthosis, epidermal inflammation, dermal inflammation, pruritus and Rosacea. Preferably the inflammatory skin disease is Netherton syndrome.

### Definitions

As used herein, the term “about” refers to a value or parameter herein that includes (and describes) embodiments that are directed to that value or parameter per se. For example, description referring to “about 50” includes description of “50.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

Medium chain triglyceride oil or medium-chain triglycerides (MCTs) are triglycerides with two or three fatty acids having an aliphatic tail of 6-12 carbon atoms, i.e., medium-chain fatty acids (MCFAs). MCFAs are; C6-caproic acid or hexanoic acid, C8-caprylic acid or octanoic acid, C10-capric acid or decanoic acid, C12-lauric acid or dodecanoic acid.

Cetostearyl alcohol, cetearyl alcohol or cetylstearyl alcohol is a mixture of fatty alcohols, consisting predominantly of cetyl (16 C) and stearyl alcohols (18 C).

Cetomacrogol 1000 is polyethylene glycol hexadecyl ether.

Isopropyl myristate (IPM) is the ester of isopropyl alcohol and myristic acid.

Butylated hydroxy toluene (BHT), also known as dibutylhydroxytoluene, is the compound 2,6-di-tert-butyl-4-methylphenol.

Polyethylene glycol (PEG) is a polyether compound with the structural formula H—(O—CH₂—CH₂)ₙ—OH.

Phenonip is a mixture of phenoxyethanol, methylparaben, ethylparaben, propylparaben, butylparaben, and isobutylparaben.

### Examples

Example 1. Testing of solubility of 6-ethoxy-7-methoxy-
2-(2-methylsulfanylphenyl)-3,1-benzoxazin-4-one (active)

| | Amount | | | | Appearance after | Appearance after |
|---|---|---|---|---|---|---|
| Exp | of active | | Vehicle^{1, 2} | Procedure | preparation | storage at RT |
| A | 7.7 | mg/g | 55% Capmul MCM | Ultrasonication + | Clear solution + | Clear solution + large |
| | | | 15% Akomed R MCT | heat | small amounts of | amounts of needle- |
| | | | 15% Cremophor RH40 | | needle-shaped | shaped crystals after |
| | | | 15% Ethanol | | crystals | 4 days |
| B | 6.3 | mg/g | 55% Capmul MCM | Ultrasonication + | Clear solution + | — |
| | | | 25% Akomed R MCT | heat | small amounts of | |
| | | | 15% NMP | | needle-shaped | |
| | | | 5% Tego SMO 80V | | crystals | |
| C | 5.9 | mg/g | 35% Capmul MCM | Heat | Clear solution | Unchanged after 1.5 |
| | | | 35% NMP | | | year |
| | | | 25% Akomed R MCT | | | |
| | | | 5% Tego SMO 80V | | | |
| D | 1.0 | mg/g | 100% MCT | Heat | Clear solution | Unchanged after 1.5 |
| | | | | | | year |
| E | 1.2 | mg/g | 100% Capmul MCM | Heat | Clear solution | Unchanged after 1.5 |
| | | | C8 EP | | | year |
| F | 50.9 | mg/g | 100% NMP | Mild heat | Clear solution | Precipitation after |
| | | | | | | 30 min |
| | 26.2 | mg/g | | Mild heat | Clear solution | Clear solution + small |
| | | | | | | amounts of needle- |
| | | | | | | shaped crystals after 1 |
| | | | | | | day. |
| G | 34.9 | mg/g | 100% Benzyl alcohol | Heat | Clear solution | Unchanged after 1.5 |
| | | | | | | year. Evaporation |
| H | 1.7 | mg/g | 66% MCT | Ultrasonication + | Clear solution | Unchanged after 1 |
| | | | 34% MCM C8 EP | heat | | month |
| I | 1.6 | mg/g | 69% MCT | Ultrasonication + | Clear solution | Unchanged after 1 |
| | | | 31% IPM | heat | | month. No visible |
| | | | | | | signs of crystals |
| | | | | | | in the microscope. |
| | | | | | | Unchanged after 1.5 |
| | | | | | | year |
| J | 1.5 | mg/g | 69% MCT | Ultrasonication + | Clear solution | Unchanged after 1.5 |
| | | | 31% Oleic acid | heat | | year |
| K | 1.9 | mg/g | 92% MCT | Ultrasonication + | Clear solution | Unchanged after 1.5 |
| | | | 8% NMP | heat | | year |
| L | 1.0 | mg/g | 100% Sesame oil | Ultrasonication + | Clear solution | Unchanged after 1.5 |
| | | | | heat | | year |
| M | 2.1 | mg/g | 100% | Ultrasonication + | Dispersion | Unchanged after 1 |
| | | | Hexamethyldisiloxane | heat | (insoluble) | month |
| N | 1.1 | mg/g | 86% Denatured alcohol | Ultrasonication + | Clear solution | Clear solution + small |
| | | | 9% PC from soybean | heat | | amounts of needle- |
| | | | 5% Lactic acid | | | shaped crystals after |
| | | | | | | 2 days |
| O | 1.25 | mg/g | 100% Mineral oil | Heat (80° C. for | Clear slightly | Sediments + cloudy |
| | | | (liquid paraffin) | about 1 h) + | brownish solution + | supernatant after 4 d. |
| | | | | magnetic stirring | large amounts of | Unchanged after 3 w |
| | | | | | needle-shaped | |
| | | | | | crystals | |
| P | 1.25 | mg/g | 67% MCT | Heat (55 → 80° C. | Clear, slightly | Unchanged after 1 m. |
| | | | 33% IPM | for about 1 h) + | brownish solution | Withstand more than 2 |
| | | | | magnetic stirring | | weeks at 5° C. |
| Q | 1.25 | mg/g | 100% MCT | Heat (80° C. for | Clear, slightly | Unchanged after 2 |
| | | | | about 15 min) + | brownish solution | years. Withstand more |
| | | | | magnetic stirring | | than 2 weeks at 5° C. |

¹All percentages are by weight
²Key to the excipients as they appear in the table:
MCM = Medium-chain monoacylglycerol
MCT = Medium-chain triacylglycerol
Cremophor RH40 = Polyoxy1 40 hydrogenated castor oil
Tego SMO 80V = Polysorbate 80
NMP = N-Methyl-2-pyrrolidone
IPM = Isopropylmyristate

Example 2. MCT based formulations

| | Amount | | | Appearance after | Appearance after |
|---|---|---|---|---|---|
| Exp | of active | Vehicle^{1, 2} | Procedure | preparation | storage at RT |
| A | 0.09 mg/g | 50% MCT | Heat (up to 70° C. | Milky low-viscous | Unchanged after 4 d |
| | | 10% IPM | for oil phase, lower | emulsion - viscous | in the refrigerator |
| | | 5% Cetostearyl alcohol | for aqueous phase) + | cream upon cooling | |
| | | 5% Cetomacrogol 1000 | high-speed mixing | | |
| | | 30% Citrate buffer, 25 | (Ultra-Turrax | | |
| | | mM, pH = 4.20 | homogenizer) | | |
| B | 0.25 mg/g | 50% MCT | Heat (up to 70° C. | Milky low-viscous | Unchanged after 4 d |
| | | 10% IPM | for oil phase, lower | emulsion - viscous | in the refrigerator |
| | | 5% Cetostearyl alcohol | for aqueous phase) + | cream upon cooling | |
| | | 5% Cetomacrogol 1000 | high-speed mixing | | |
| | | 30% Citrate buffer, 25 | (Ultra-Turrax | | |
| | | mM, pH = 4.20 | homogenizer) | | |
| C | 0.75 mg/g | 50% MCT | Heat (up to 70° C. | Milky low-viscous | Unchanged after 4 d |
| | | 10% IPM | for oil phase, lower | emulsion - viscous | in the refrigerator |
| | | 5% Cetostearyl alcohol | for aqueous phase) + | cream upon cooling | |
| | | 5% Cetomacrogol 1000 | high-speed mixing | | |
| | | 30% Citrate buffer, 25 | (Ultra-Turrax | | |
| | | mM, pH = 4.20 | homogenizer) | | |
| D | 0.75 mg/g | 40% MCT | Heat (80° C. for | Milky low-viscous | Unchanged after 3 w |
| | | 20% IPM | about 1 h for oil | emulsion - viscous | |
| | | 5% Cetostearyl alcohol | phase, shorter for | cream upon cooling | |
| | | 5% Cetomacrogol 1000 | aqueous phase) + | | |
| | | 30% Citrate buffer, 25 | high-speed mixing | | |
| | | mM, pH = 4.20 | (Ultra-Turrax | | |
| | | | homogenizer) | | |
| E | 2.24 mg/g | 40% MCT | Heat (80° C. for | Milky low-viscous | Unchanged after 1 d. |
| | | 20% IPM | about 30 min for | emulsion - viscous | Withstand |
| | | 5% Cetostearyl alcohol | oil phase, shorter | cream upon cooling | centrifugation at |
| | | 5% Cetomacrogol 1000 | for aqueous phase) + | | 4000 rpm for 2 min |
| | | 30% Citrate buffer, 25 | high-speed mixing | | without separation |
| | | mM, pH = 4.20 | (Ultra-Turrax | | |
| | | | homogenizer) | | |

Example 3. Preparation of cream - batch size 100 g

| Oil phase: | | | |
|---|---|---|---|
| MCT | 50.0% | (w/w) | Solubilizer and emollient |
| IPM | 10.0% | (w/w) | Emollient |
| Cetostearyl alcohol | 5.0% | (w/w) | Thickener |
| Cetomacrogol 1000 | 5.0% | (w/w) | Emulsifier |
| BHT | 0.1% | (w/w) | Antioxidant |
| 6-Ethoxy-7-methoxy-2-(2- | 0.075% | (w/w) | Active pharmaceutical |
| methylsulfanylphenyl)- | | | ingredient |
| 3,1-benzoxazin-4-one | | | |
| Aqueous phase: | | | |
| Potassium sorbate | 0.20% | (w/w) | Preservative |
| Sorbic acid | 0.15% | (w/w) | Preservative |

| | |
|---|---|
| Citrate buffer, 25 mM, pH = 4.20 | ad 100% |

The oily ingredients were weighed and mixed in a beaker. The aqueous phase was mixed in another beaker. The oil phase and the aqueous phase were both heated to about 75° C. while stirring. The warm aqueous phase was added to the warm oil phase during high-shear mixing (Ultra-Turrax® or the like). The emulsification (high-shear mixing) continued intermittently until the temperature had reached about 30° C. and a homogeneous white viscous cream was formed.

Example 4. Preparation of cream - batch size 40 kg

| Oil phase: | | | |
|---|---|---|---|
| MCT | 50.0% | (w/w) | Solubilizer and emollient |
| IPM | 5.0% | (w/w) | Emollient |
| Cetostearyl alcohol | 7.5% | (w/w) | Thickener |
| Cetomacrogol 1000 | 7.5% | (w/w) | Emulsifier |
| BHT | 0.1% | (w/w) | Antioxidant |
| 6-Ethoxy-7-methoxy-2-(2- | 0.075% | (w/w) | Active pharmaceutical |
| methylsulfanylphenyl)- | | | ingredient |
| 3,1-benzoxazin-4-one | | | |
| Aqueous phase: | | | |
| Potassium sorbate | 0.20% | (w/w) | Preservative |
| Sorbic acid | 0.15% | (w/w) | Preservative |

| | |
|---|---|
| Citrate buffer, 25 mM, | ad 100% |
| pH = 4.20 | |

The oily ingredients were weighed and mixed in a 50-litre Olsa® tank. The aqueous phase was mixed in a 15-litre beaker. The oil phase and the aqueous phase were both heated to about 70° C. while stirring. The warm aqueous phase was added to the warm oil phase during high-shear mixing. The equipment was then put under vacuum. The emulsification (high-shear mixing) continued at 3,000 rpm for 30 min. Cooling was then performed during slow mixing until the temperature had reached about 25° C. and a homogeneous white viscous cream was formed. Finally, the cream was filled in 10-ml aluminium tubes.

1. A topical formulation being an oil-in-water emulsion, said formulation comprising,
a) from about 0.01% (w/w) to about 0.2% (w/w) 6-ethoxy-7-methoxy-2-(2-methylsulfanylphenyl)-3,1-benzoxazin-4-one,
b) from about 60% (w/w) to about 75% (w/w) oil phase, and
c) from about 25% (w/w) to about 40% (w/w) aqueous phase.

2. The topical formulation according to claim 1, wherein the oil phase comprises more than 50% (w/w) medium chain triglyceride oil (MCT).

3. The topical formulation according to claim 1, said formulation comprising
a) about 0.075% (w/w) 6-ethoxy-7-methoxy-2-(2-methylsulfanylphenyl)-3,1-benzoxazin-4-one,
b) about 50% (w/w) medium chain triglyceride oil (MCT),
c) about 10% (w/w) isopropyl myristate (IPM),
d) about 5% (w/w) cetostearyl alcohol,
e) about 5% (w/w) polyethylene glycol hexadecyl ether, and
f) about 30% (w/w) citrate buffer.

4. The topical formulation according to claim 1, said formulation comprising,
a) about 0.075% (w/w) 6-ethoxy-7-methoxy-2-(2-methylsulfanylphenyl)-3,1-benzoxazin-4-one,
b) about 50% (w/w) medium chain triglyceride oil (MCT),
c) about 5% (w/w) isopropyl myristate (IPM),
d) about 7.5% (w/w) cetostearyl alcohol,
e) about 7.5% (w/w) polyethylene glycol hexadecyl ether, and
f) about 30% (w/w) citrate buffer.

5. The topical formulation according to claim 1 for use in prophylaxis, prevention, and/or treatment of a skin disease.

6. The topical formulation according to claim 1 for use in prophylaxis, prevention, and/or treatment of an inflammatory skin disease.

7. The topical formulation according to claim 1 for use in prophylaxis, prevention, and/or treatment of an inflammatory skin disease selected from Netherton syndrome, atopic dermatitis, contact dermatitis, eczema, psoriasis, acne, epidermal hyperkeratosis, acanthosis, epidermal inflammation, dermal inflammation, and pruritus and Rosacea.

8. The topical formulation according to claim 7, wherein the inflammatory skin disease is Netherton syndrome.
